(19) United States
(12) Patent Application Publication
(10) Pub. No.: US 2013/0143753 A1
(43) Pub. Date: Jun. 6, 2013

(54) METHODS FOR PREDICTING OUTCOME OF BREAST CANCER, AND/OR RISK OF RELAPSE, RESPONSE OR SURVIVAL OF A PATIENT SUFFERING THEREFROM

(71) Applicants: Abderrahim El Guerrab, Clermont-Ferrand (FR); Anne Cayre, Clermont-Ferrand (FR); Fabrice Kwiatkowski, Clermont-Ferrand (FR); Maud Privat, Clermont-Ferrand (FR); Jean-Marc Rossignol, Clermont-Ferrand (FR); Fabrice Rossignol, Clermont-Ferrand (FR); Frederique Penault Llorca, Clermont-Ferrand (FR); Yves Jean Bignon, Clermont-Ferrand (FR)

(72) Inventors: Abderrahim El Guerrab, Clermont-Ferrand (FR); Anne Cayre, Clermont-Ferrand (FR); Fabrice Kwiatkowski, Clermont-Ferrand (FR); Maud Privat, Clermont-Ferrand (FR); Jean-Marc Rossignol, Clermont-Ferrand (FR); Fabrice Rossignol, Clermont-Ferrand (FR); Frederique Penault Llorca, Clermont-Ferrand (FR); Yves Jean Bignon, Clermont-Ferrand (FR)

(73) Assignees: ADELBIO, AUBIERE (FR); CENTRE JEAN PERRIN CENTRE DE LUTTE CONTRE LE CANCER DE LA REGION AUVERGNE, CLERMONT-FERRAND (FR); UNIVERSITE D'AUVERGNE CLERMONT I, CLERMONT FERRAND (FR)

(21) Appl. No.: 13/582,388

(22) PCT Filed: Mar. 1, 2010
(86) PCT No.: PCT/IB10/01108

§ 371 Date: Feb. 19, 2013

(51) Int. Cl. C12Q 1/68

(52) U.S. Cl. CPC C12Q 1/6886
USPC 506/9; 506/16

(57) ABSTRACT

The present invention relates to biomarkers allowing predicting breast tumor and solid tumor outcome using hypoxia related genes. More specifically, the present invention relates to a method for predicting the survival of a patient suffering from cancer, said method comprising the steps of (a) measuring the expression of at least five genes selected from the group consisting of GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK1 9, VIM, CXCR4, UPAR, CATHD, CTGF, C0X2, MET, IGF-2, CCND1, EPO, NDRG1, BNIP3, NIX, ETS1, PHD2, TWIST1, DEC1, SNAH, CEBPA, CITED2, F0X03A, NUR77, BRCA1, PTEN, VHL and ERBB2 in a biological sample of said patient, and (b) analyzing the expression values to generate a risk score of relapse, wherein a risk score superior or equal to three is indicative of high risk of relapse and a risk score inferior or equal to two is indicative of a low risk of relapse. In particular the following genes: EPO, ETS1, ENO1, PGK1, LDHA, TPI and optionally VEGFA were significantly over-expressed in patients with relapse.

The present invention relates to biomarkers allowing predicting breast tumor and solid tumor outcome using hypoxia related genes. More specifically, the present invention relates to a method for predicting the risk of relapse of a patient suffering from breast cancer, said method comprising the steps of (a) measuring the expression of at least five genes selected from the group consisting of GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2 in a biological sample of said patient, and (b) analyzing the expression values to generate a risk score of relapse, wherein a risk score superior or equal to two is indicative of high risk of relapse and a risk score inferior to three is indicative of a low risk of relapse.

### BACKGROUND OF THE INVENTION

Solid tumor cells generally grow in a hypoxic environment. Oxygen partial pressure is usually lower than in normal tissue (0-20 mmHg vs. 20-65 mmHg, respectively). This is partially due to abnormal vascularization of tumors, said vascularization being insufficient for supplying oxygen to the expanding tumor cells. Hypoxia triggers a multitude of cellular processes, including angiogenesis, drug resistance, cell survival, cell mobility, matrix remodeling, proliferation, differentiation, synthesis of growth factor, and changes in glucose, pH and iron metabolism. Tumors with severe hypoxic stress or necrosis have been recognized as being more resistant to conventional treatments than other tumors.

They are thus associated with a poorer prognostic for response, outcome and survival.

The above cellular processes are mainly controlled by the Hypoxia Inducible Factor (HIF-1). This factor is a heterodimeric basic helix-loop-helix transcription factor comprising two subunits: HIF-1α and HIF-1β (Wang and Semenza 1995 J Biol Chem 270:1230-7). HIF-1 recognizes DNA sequences referred to as hypoxia response elements (HRE), and induces the transcription of target genes, including vascular endothelium growth factor, erythropoietin and glycolysis isozymes. In hypoxic conditions, HIF-1α accumulates whereas in normoxia, the tumor suppressor VHL (von Hippel-Lindau) targets ubiquitinylated HIF-1α to proteasomal degradation (Maxwell et al. 1999 Nature 399:271-5). This ubiquitinylation is only seen after hydroxylation of specific proline residues (Pro402, Pro564), and is catalyzed by an O2-regulated prolyl hydroxylase (Ivan et al. 2001 Science 292: 464-8; Jaakkola et al. 2001 Science 292:468-72; McNeill et al. 2002 Bioorg Med Chem Lett 12:1547-50; Hon et al. 2002 Nature 5:5; Cioffi et al. 2003 Biochem Biophys Res Commun 303:947-53). The regulation of HIF-1 transactivation involves asparagine hydroxylation, which is catalyzed by an O₂- and Fe(II)-dependent asparaginyl hydroxylase previously identified as Factor Inhibiting HIF-1 (Lando et al. 2002 Science 295:858-61; Sang et al. 2002 Mol Cell Biol 22:2984-92; Hewitson et al. 2002 J Biol Chem 31:31; Masson and Ratcliffe 2003 J Cell Sci 116:3041-9).

On the other hand, HER2 (neu) signaling increases the rate of HIF-1a synthesis and provides a mechanism for HIF-1-mediated vascular endothelial growth factor expression (Laughner et al. 2001 Mol Cell Biol 21:3995-4004).

Breast cancer is a heterogeneous disease and there is a continual drive to identify markers that will aid in predicting prognosis and response to therapy. To date, relatively few markers have established prognostic power. Oestrogen receptor (ER) is one predictive marker in breast cancer management, useful both for determining prognosis and for predicting response to hormone therapies. Progesterone receptor (PR) is also a widely used marker, although its value is less well established. HER-2 status has also become a routine prognostic and predictive factor in breast cancer.

Given the importance of these biological markers in patient management, it is essential to identify other such markers, and to obtain a robust, sensitive and reliable method for predicting outcome, survival and response of patients.

One molecular diagnostic test, marketed by Agendia under the name of MammaPrint®, is currently available for assessing the risk that a breast tumor will spread to other parts of the body. This diagnostic test is based on a 70-gene breast cancer gene signature (Van't Veer et al. 2002 Nature 415: 530-6). MammaPrint® is based on the use of microarrays, and aims at assessing the risk of developing metastases. It is mainly used for assessing the risk of developing metastases in patients suffering from lymph node negative breast cancers. In particular, MammaPrint® does not allowing assessing the risk of relapse.

The OncoTypeDX® diagnostic test allows assessing the risk of relapse but is based on the analysis of as many as twenty-one different genes.

There is therefore a need in the art for markers allowing predicting the outcome of a patient suffering from breast cancer. In particular, there is a need in the art for a set of markers, comprising a few markers only, which allows accurately assessing the risk of relapse.

### DETAILED DESCRIPTION OF THE INVENTION

The inventors have surprisingly found that hypoxia related genes can be used as biomarkers for predicting outcome of breast cancer, and/or for predicting survival and/or response of a patient suffering from breast cancer. In particular, the GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2 hypoxia related genes can be used for this purpose. Measuring the expression of these genes allows distinguishing subgroups of cancers, e.g. cancers for which the risk of relapse is high and cancers for which the risk of relapse is low.

In particular, it has been found that analysis of the expression values of the EPO, ETS1, ENO1, PGK1, LDHA and TPI genes allows generating a risk score of relapse, wherein a risk score having a value 3 is indicative of a short relapse time and a risk score having a value 2 is indicative of a long relapse time.

The inventors have demonstrated by in silico studies that sustained hypoxia related gene transactivation in HER and antiangiogenic treatments are associated with resistance to treatment and poorer outcome. Gene response in solid tumors was predicted by in silico studies, in vitro assays and bibliographic data. An optimized combination of hypoxia-related genes was determined. This optimized combination of hypoxia-related genes allows predicting the fate of breast cancer cells and the response to conventional and targeted therapies.

The invention therefore entails measuring the differential expression of a plurality of relapse-related biomarkers in a sample of cells from a patient suffering from cancer, said plurality of relapse-related biomarkers comprising at least some of the biomarkers identified by the inventors. The differential pattern of expression in each cancer—or gene expression signature—may then be used to generate a risk score that is predictive of relapse.

The present invention thus provides a set of biomarkers allowing predicting the outcome of cancer, preferably of breast cancer. More specifically, the present invention relates to methods for predicting treatment responses of an individual suffering from cancer, preferably breast cancer, and to methods for assigning a prognostic score to non-treated patients based on biopsies obtained therefrom. It also concerns a set of hypoxic response genes useful for designing first line treatment, anti-HER2 and anti-angiogenesis treatments for a patient suffering from cancer, preferably breast cancer.

1. HypBiomarkers According to the Invention

The biomarkers identified by the inventors, namely GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2 genes will further be referred to as the “HypBiomarkers”.

The HypBiomarkers according to the invention are defined in Table 1 herebelow. This table provides the name of the gene, an accession number allowing identifying the sequence of one or more messenger RNA(s) encoded by the gene, as well as a brief description of the encoded protein. The sequence corresponding to the accession number can be found in the NCBI database (World Wide Web site ncbi.nlm.nih.gov). The version of the sequence preferably corresponds to the version in force on Dec. 1, 2009.

As used herein the term “gene” refers to the nucleotidic sequence including the 5′ regulatory region, the promoter, the introns, the exons and the 3′ regulatory region. As known to one skilled in the art, a gene includes both transcribed and untranscribed regions. The transcribed region may include introns, which are spliced out of the mRNA, and 5′- and 3′-untranslated (UTR) sequences along with the protein coding sequences (exons).

As used herein, the term “messenger RNA (mRNA)” refers to a nucleotidic sequence transcribed from a gene, i.e. a sequence lacking introns. As known to one skilled in the art, several different mRNAs may be transcribed from a given gene depending on alternative splicing. In the frame of the present invention, the term mRNA is meant to encompass all alternative splice variants of a gene. In a specific embodiment, the mRNA corresponds to an mRNA, the sequence of which is listed in Table 1.

In the frame of the present invention, the HypBiomarkers include all allelic variants of the genes listed in Table 1.

TABLE 1

| | Alternative | | |
|---|---|---|---|
| Gene Name | names | Accession Number | Encoded protein |
| GLUT1 | SLC2A1 | NM_006516.2 | Glucose Transporter 1 |
| PGK1 | PGKA | NM_000291.3 | PhosphoGlycerate Kinase 1 |
| LDHA | LDH-M | NM_005566.2 | Lactate DeHydrogenase A |
| | LDH1 | | |
| ENO1 | ENO1L1 | NM_001428.2 | Enolase 1 |
| | MBP-1 | | |
| | MPB1 | | |
| CAIX | CA9 | NM_001216.1 | Carbonic Anhydrase 9 |
| NHERF1 | SLC9A3R1 | NM_004252.2 | Solute carrier family 9 |
| | | | (sodium/hydrogen exchanger), member |
| | | | 3 regulator 1 |
| TPI | TP1 | NM_000365.4 | Triose-Phosphate Isomerase |
| AMF/GPI | | NM_000175.2 | Glucose Phosphate Isomerase |
| VEGFA | | NM_001025366.1, | Vascular Endothelial Growth Factor |
| | | NM_001025367.1, | |
| | | NM_001025368.1, | |
| | | NM_001025369.1, | |
| | | NM_001025370.1, | |
| | | NM_001025366.1, | |
| | | NM_001033756.1, | |
| | | NM_003376.4 | |
| TGFB3 | | NM_003239.2 | Transforming Growth Factor Beta |
| ENG | CD105 | NM_000118.1, | Endoglin |
| | END | NM_001114753.1 | |
| LEP | | NM_000230.2 | Leptin |
| EDN1 | ET1 | NM_001955.3 | Endothelin |
| MDR1 | ABCB1 | NM_000927.3 | Multi-Drogue Resistance 1 |
| AK3 | | NM_016282.2 | Adenylate Kinase |
| MXR1 | ABCG2 | NM_004827.2 | ATP-binding cassette, sub-family G |
| | | | (WHITE), member 2 |
| TGM2 | | NM_0046132, | Transglutaminase 2 |
| | | NM_198951.1 | |
| CDH1 | CDHE | NM_004360.3 | E-Cadherin |
| MMP2 | | NM_004530.2 | Matrix Metallopeptidase 2 |
| CK19 | KRT19 | NM_002276.4 | Keratin 19 |
| VIM | | NM_003380.2 | Vimentin |
| CXCR4 | NPYR | NM_001008540.1, | Chemokine (C—X—C motif) receptor 4 |
| | | NM_003467.2 | |
| UPAR | PLAUR | NM_001005376.1, | Plasminogen Activator, Urokinase |
| | | NM_001005377.1, | Receptor |
| | | NM_002659.2 | |
| CATHD | CTSD | NM_001909.3 | Cathepsin D |
| CTGF | | NM_001901.2 | Connective tissue growth factor |
| COX2 | PTGS2 | NM_000963.1 | Prostaglandin-endoperoxide synthase 2 |
| | | | (prostaglandin G/H synthase and |
| | | | cyclooxygenase) |
| MET | | NM_000245.2 | proto-oncogene MET product |
| IGF2 | | NM_001007139.4, | Insulin-like Growth Factor 2 |
| | | NM_000612.4 | (somatomedin A) |
| CCND1 | | NM_053056.2 | Cyclin D1 |
| EPO | | NM_000799.2 | Erythropoietin |
| NDRG1 | | NM_006096.2 | N-myc Downstream Regulated Gene 1 |
| BNIP3 | | NM_004052.2 | BCL2/adenovirus E1B 19 kd-interacting |
| | | | protein 3 |
| NIX | BNIP3L | NM_004331.2 | BCL2/adenovirus E1B 19 kDa |
| | | | interacting protein 3-like |
| ETS1 | | NM_005238.2 | v-ets erythroblastosis virus E26 |
| | | | oncogene homolog 1 |
| PHD2 | EGLN1 | NM_022051.1 | HIF-prolyl hydroxylase 2 |
| TWIST1 | | NM_000474.3 | Twist homolog 1 |
| | | | (acrocephalosyndactyly 3; Saethre- |
| | | | Chotzen syndrome) |
| SNAI1 | | NM_005985.2 | Snail homolog 1 |
| CEBPA | | NM_004364.2 | CCAAT/Enhancer Binding Protein |
| | | | Alpha |
| CITED2 | | NM_006079.3 | Cbp/p300-interacting transactivator, |
| | | | with Glu/Asp-rich carboxy-terminal |
| | | | domain, 2 |
| FOXO3A | FOXO3 | NM_201559.2, | Forkhead box O3 |
| | | NM_001455.3 | |
| NUR77 | NR4A1 | NM_173157.1, | Nuclear receptor subfamily 4, group A, |
| | | NM_002135.3 | member 1 |
| BRCA1 | | NM_007294.2, | Breast Cancer 1 |
| | | NM_007295.2, | |
| | | NM_007296.2, | |
| | | NM_007297.2, | |
| | | NM_007298.2, | |
| | | NM_007299.2, | |
| | | NM_007300.2, | |
| | | NM_007302.2, | |
| | | NM_007303.2, | |
| | | NM_007304.2, | |
| | | NM_007305.2 | |
| PTEN | | NM_000314.4 | Phosphatidylinositol-3,4,5- |
| | | | Trisphosphate 3-Phosphatase |
| VHL | | NM_000551.2 | Von Hippel-Lindau |
| ERBB2 | HER-2/neuc- | NM_001005862.1, | Erythroblastic leukemia viral oncogene |
| | erb B2 | NM_004448.2 | homolog 2 |

In a preferred embodiment, the HypBiomarkers according to the invention comprise or consist of EPO, ETS1, ENO1, PGK1 LDHA, TPI, and optionally VEGFA.

2. Methods According to the Invention

An aspect of the invention is directed to a method for predicting the risk of relapse of a patient suffering from cancer, preferably breast cancer, said method comprising the steps of:
a) measuring the expression of at least five of the HypBiomarkers according to the invention in a biological sample of said patient; and
b) analyzing the expression values to generate a risk score of survival;
   wherein a risk score superior or equal to three is indicative of high risk of relapse and a risk score inferior or equal to two is indicative of a low risk of relapse.

The expression value of each biomarker is determined, and the expression values are analyzed to generate a prognostic score or treatment susceptibility score. A risk score superior or equal to three is indicative of high risk of relapse and a risk score inferior or equal to two is indicative of a low risk of relapse.

In a preferred embodiment, the at least five HypBiomarkers comprise or consist of EPO, ETS1, ENO1, PGK1 LDHA, TPI, and optionally VEGFA. Most preferably, the risk score is generated as described in Example 1. Briefly, the risk score is calculated as follows:
measuring the expression of EPO, ETS1, ENO1, PGK1 LDHA, TPI in a biological sample of said patient;
normalizing the expression of said genes in order to obtain a value. Said value may for example correspond to the relative quantity (RQ) calculated using the equation RQ=2−ΔΔCt;
for each gene, comparing said value to the reference value shown in Table 2 of Example 2;
for each gene, attributing the number of 1 if its expression is higher than the reference value presented shown in Table 2, and the number 0 if its expression is lower; and
making the sum of the numbers attributed to each gene.

Another aspect of the invention is directed to a method for predicting outcome of cancer, preferably breast cancer, and/or for predicting survival and/or response of a patient suffering from cancer, preferably breast cancer, said method comprising the steps of:
a) measuring the expression of at least five of the HypBiomarkers according to the invention in a biological sample of a patient; and
b) correlating the expression measured at step (a) with outcome, survival and/or response.
   Step (b) allows deducing the outcome of said cancer, and/or the survival and/or the response of said patient.

Correlating the expression measured at step (a) with outcome, survival and/or response can be done, e.g., by comparing the expression of a HypBiomarker in the biological sample to a reference value, wherein a significant higher or lower level of expression in the biological sample, compared to the reference value, is an indication for the outcome of said cancer, and/or the survival and/or the response of said patient.

The methods according to the invention are not carried out in vivo, but in vitro and/or ex vivo.

As used herein, the term “cancer” refers to any malignant solid tumor. Indeed, hypoxia arises in all solid tumor. Thus the methods according to the invention may be used to predict outcome, survival and/or response of a patient suffering from any type of malignant solid tumor.

In a preferred embodiment of the invention, the cancer is a breast cancer. The term “breast cancer” refers to any type of malignant tumor of the breast. Most breast cancers are epithelial tumors that develop from cells lining ducts or lobules; less common are nonepithelial cancers of the supporting stroma (e.g. angiosarcoma, primary stromal sarcomas, phyllodes tumor). In the frame of the present invention, the breast cancer is preferably a breast carcinoma or a breast adenocarcinoma (e.g. a ductal carcinoma in situ, infiltrating ductal carcinoma, invasive ductal breast carcinoma, medullary carcinoma, mucinous carcinoma, tubular carcinoma or Paget's disease of the nipple). Most preferably, it is an infiltrating ductal carcinoma or invasive ductal breast carcinoma.

As used herein, the term “patient suffering from cancer” referred to an individual, preferably a human individual, diagnosed with cancer. In a specific embodiment, the patient is a newly diagnosed individual, i.e. an individual that has not yet been treated for cancer.

Step (a) of the methods according to the invention preferably comprises or consists of the measure of the expression of at least 5, 10, 15, 20, 25, 30, 35, 40 or 45 HypBiomarkers. In a preferred embodiment, expression of all HypBiomarkers is measured.

Step (a) of the methods according to the invention may further comprise the measure of the expression of calibration genes such as, e.g., genes encoding the ribosomal protein L32 (RPL32; Accession Nos. NM_{—}001007073.1, NM_{—}001007074.1 and/or NM_{—}000994.3) and/or the 18S rRNA. Such calibration genes are useful for normalizing the expression levels of the different HypBiomarkers.

Step (a) of the methods according to the invention may further comprise the measure of the expression of additional genes known to be predictive for the outcome, survival and/or response of cancer, preferably breast cancer, or of a patient suffering therefrom.

As used herein, the term “predicting” a status or event refers to making a finding that an individual has a significantly enhanced or reduced probability of having a given status or experiencing an event. In particular, the present invention aims at predicting:
the outcome of cancer, preferably breast cancer (i.e. the condition of a patient at the end of treatment, such as e.g. death, partial remission, complete remission, remission with or without a risk of relapse, etc.);
the survival of a patient (i.e. the length of time that a person lives after being diagnosed with beast cancer, such as e.g. five-year or ten-year survival, event-free survival, progression-free survival, etc.);
the response of a patient (i.e. a positive medical response to a treatment).

In particular, it has been shown that the methods according to the invention allow predicting the grade of the cancer, the probability of the patient to relapse, and proteins expressed by the tumor cells. This information allows in turn predicting the outcome, survival and response. For example, a patient suffering from a beast cancer predicted to be of high grade is likely to have a bad outcome and a poor survival.

In a preferred embodiment, predicting outcome of cancer comprises or consists of predicting the risk of relapse. Indeed, the methods according to the invention allow predicting whether a patient is likely to relapse or not. In a most preferred embodiment, the methods are carried out using the EPO, ETS1, ENO1, PGK1, TPI, LDHA and optionally VEGFA HypBiomarkers, and a significant overexpression of at least three of EPO, ETS1, ENO1, PGK1, TPI, LDHA and optionally VEGFA in a patient suffering from cancer, preferably breast cancer, compared to a reference value, indicates that said patient is likely to relapse.

The methods according to the invention also allow predicting the grade of the cancer, preferably breast cancer. Several classifications allowing determining the grade of a cancer are well-known to the skilled in the art. Such classifications include the Scarff Bloom and Richardson (SBR) classification (Scarf and Torloni 1968 “Histological typing of breast tumors” in International histological classification of tumours, no. 2. Vol. 2. World Health Organization, Geneva, pp. 13-20; Bloom, and Richardson 1957 Br. J. Cancer 11:359-377) and the modified Scarf Bloom and Richardson (mSBR) classification (Elston and Ellis 1991 Histopathology 19:403-10). Cancers of high grade correspond to aggressive cancers, i.e. to cancers that grow quickly and tend to spread rapidly.

In one embodiment, the methods are carried out using the NHERF1, ERBB2, PTEN, MET and CDH1 HypBiomarkers, and a significant overexpression of NHERF1, ERBB2, PTEN, MET and CDH1 in a patient suffering from cancer, preferably breast cancer, compared to a reference value, indicates that said cancer is likely to be of grade 2 or grade 3 according to the Scarf Bloom and Richardson (SBR) classification that the cancer is likely to be an aggressive cancer.

In another embodiment, the methods are carried out using the NHERF1, ERBB2, PTEN, BRCA1 and CDH1 HypBiomarkers, and a significant overexpression of NHERF1, ERBB2, PTEN, BRCA1 and CDH1 in a patient suffering from cancer, preferably breast cancer, compared to a reference value, indicates that said cancer is likely to be of grade 4 or 5 according to the modified Scarff Bloom and Richardson (mSBR) classification, i.e., that the cancer is likely to be an aggressive cancer.

In still another embodiment, the methods are carried out using the NHERF1, ERBB2, PTEN, CDH1, MET and BRCA1 HypBiomarkers, and a significant overexpression either of NHERF1, ERBB2, PTEN, MET and CDH1 or of NHERF1, ERBB2, PTEN, BRCA1 and CDH1 in said patient, compared to a reference value, indicates that said cancer is likely to be an aggressive cancer

The method according to the invention further allow predicting whether a patient suffering from cancer, preferably breast cancer, express proteins relevant for designing a treatment regimen for the patient, such as e.g. the estrogen receptor (ER), the progesterone receptor (PR), or HER2/neu proteins.

In one embodiment, the methods are carried out using the CATHD, MDR1, NIX, FOXO3A, ETS1, TWIST1, VHL, IGF2, PHD2 and EDN1 HypBiomarker, and a significant overexpression of CATHD, MDR1, NIX, FOXO3A, ETS1, TWIST1, VHL, IGF2, PHD2 and EDN1 in a patient suffering from cancer, preferably breast cancer, compared to a reference value, indicates that the cancer is likely to express progesterone receptors, i.e. is likely to respond to therapies targeting progesterone receptors.

In another embodiment, the methods are carried out using the ERBB2, NHERF1, CDH1, TPI, CITED2, KRT19 and GLUT1 HypBiomarker, and a significant overexpression of ERBB2, NHERF1, CDH1, TPI, CITED2, KRT19 and GLUT1 in a patient suffering from cancer, preferably breast cancer, compared to a reference value, indicates that the cancer is likely to be a Her2-positive cancer, i.e. is likely to respond to therapies targeting HER receptors.

In still another embodiment, the methods are carried out using the ERBB2, NHERF1, COX2, TWIST1, PHD2, PGK1, FOXO3A, TGM2, CDH1, CATHD and EDN1 HypBiomarker, and a significant overexpression of ERBB2, NHERF1, COX2, TWIST1, PHD2, PGK1, FOXO3A, TGM2, CDH1, CATHD and EDN1 in a patient suffering from cancer, preferably breast cancer, compared to a reference value, indicates that the cancer is likely to be a Her2-positive 2+3+ cancer, i.e. is likely to respond to therapies targeting HER receptors.

In still another embodiment, the methods are carried out using the ERBB2, NHERF1, COX2, PHD2, PGK1, FOXO3A, TPI, MET, PTEN, ENG and GLUT1 HypBiomarker, and a significant overexpression of ERBB2, NHERF1, COX2, PHD2, PGK1, FOXO3A, TPI, MET, PTEN, ENG and GLUT1 in a patient suffering from cancer, preferably breast cancer, compared to a reference value, indicates that the cancer is likely to be a Her2-positive 3+ cancer, i.e. is likely to respond to therapies targeting HER receptors.

In still another embodiment, the methods are carried out using the ERBB2, NHERF1, CDH1, TPI, CITED2, KRT19, COX2, TWIST1, PHD2, PGK1, FOXO3, TGM2, CATHD, EDN1, MET, PTEN, ENG and GLUT1, and a significant overexpression of:
i. ERBB2, NHERF1, CDH1, TPI, CITED2, KRT19 and GLUT1;
ii. ERBB2, NHERF1, COX2, TWIST1, PHD2, PGK1, FOXO3, TGM2, CDH1, CATHD and EDN1; or
iii. ERBB2, NHERF1, COX2, PHD2, PGK1, FOXO3, TPI, MET, PTEN, ENG and GLUT1;
   in said patient, compared to a reference value, indicates that said cancer is likely to respond to therapies targeting HER receptors receptors.

As used herein, a HypBiomarker is “significantly expressed, over-expressed or under-expressed” if the p-value is inferior or to 0.10.

The “reference value” is established by statistical analysis of values obtained from a representative panel of individuals. The panel may for example depend from the nature of the sample, the age and/or sex of the individual, etc. It may be predetermined to the measure of the expression of the HypBiomarkers in the patient. The reference value can for example be obtained from a panel healthy individual, or from a panel of individuals for which the outcome, response or survival is known, or, when the methods according to the invention aim at monitoring a patient, from the patient previously tested.

Another aspect of the invention is to provide a method for screening molecule for treating patients suffering from cancer, preferably breast cancer, comprising measuring the expression of at least five HypBiomarkers. In other words, the invention relates to a method for screening molecule for treating cancer comprising the analysis of the action of said molecule on at least 5 of the HypBiomarkers.

By <<the action of said molecule>>, in the sense of the present invention, is meant the positive effect of the molecule on the survival of the patient, or on the RFS of the patient, the reduction of size of the tumor, or the diminution of the expression of the HypBiomarkers.

The methods according to the invention also allow predicting response of a patient to therapies, e.g. therapies targeting HER receptors and/or VEGF receptors. Such treatments include for example treatments with E7070, PHA-533533, hymenialdisine, NU2058 & NU6027, AZ703, BMS-387032, CYC202 (R-roscovitine), CDKi277, NU6140, PNU-252808, RO-3306, CVT-313, SU9516, Olomoucine, ZK-CDK (ZK304709), JNJ-7706621, PD0332991, PD0183812, Fascplysin, CA224, CINK4, caffeine, pentoxifylline, wortmannin, LY294002, UCN-01, debromohymenialdisine, Go6976, SB-218078, ICP-1, CEP-3891, TAT-S216A, CEP-6367, XL844, PD0166285, BI2536, ON01910, Scytonemin, wortmannin, HMN-214, cyclapolin-1, hesperadin, JNJ-7706621, PHA-680632, VX-680 (MK-0457), ZM447439, MLN8054, R763, AZD1152, CYC116, SNS-314, MKC-1693, AT9283, quinazoline derivatives, MP235, MP529, cincreasin, SP600125 (de Career et al. Current Medicinal Chemistry 2007, 14:1-17; Malumbres et al. Current Opinion in genetics & Development 2007, 17:60-65; Malumbres et al. Clin. Transl. Oncol. 2006, 8:1), Iressa (gefitnib, ZD1839, anti-EGFR, PDGFR, c-kit), ABX-EGFR (anti-EGFR, Abgenix/Amgen), Zamestra (FTI, J & J/Ortho-Biotech,; Herceptin (anti-HER2/neu, Genentech), Avastin (bevancizumab, anti-VEGF antibody, Genentech), Tarceva (ertolinib, OSI-774, RTK inhibitor, Genentech-Roche), ZD66474 (anti-VEGFR, Astra-Zeneca), Erbitux (IMC-225, cetuximab, anti-EGFR, Imclone/BMS), Oncolar (anti-GRH, Novartis); PD-183805 (RTK inhibitor, Pfizer), EMD72000 (anti-EGFR/VEGF ab, Merck KgaA), Cl-1033 (HER2/neu & EGF-R dual inhibitor, Pfizer), EGF10004, Herzyme (anti-HER2 ab, Medizyme Pharmaceuticals), Microsphere delivery of HER2/neu vaccine (Medarex), ZM447439 (AstraZeneca), MK0457 (Merck), AZD1152 (AstraZeneca), PHA-680632, MLN8054 (Millenium Pharmaceutical), PHA739358 (Nerviano Sciences), scytonemin, BI2536, ON01910 and the drugs listed in Awada et al. (Critical Reviews in Oncology/Hematology 2003 48:45-63).

The methods according to the invention may further comprise the step of designing a treatment regimen for the patient. Indeed, the above methods provide useful information regarding therapeutic genes which are expressed or not in the tumor. For example, if the cancer is likely to be a Her2-positive cancer expressing progesterone receptors, a treatment targeting HER receptors and/or VEGF receptors may be designed. If the patient has a high risk to relapse, one may opt for a treatment by an aggressive combination therapy.

The methods according to the invention are also useful for monitoring progression of cancer, preferably breast cancer, and/or for monitoring the efficiency of a treatment. In such a case, the methods described hereabove further comprises the step of repeating steps (a) and (b) on another biological sample of the same patient at a later point in time. It can thus be determined whether the second prognostic of the patient is better, unchanged or worse than the first prognostic. The biological samples may for example have been taken before and after a treatment, respectively.

Measuring the expression of the HypBiomarkers according to the invention can be done by a variety of techniques that are well-known to the skilled in the art. Such techniques typically include methods based on the determination of the level of transcription (i.e. the amount of mRNA produced) and methods based on the quantification of the protein encoded by the genes. Measuring the expression of a gene preferably includes quantifying the expression of said gene. Information regarding methods for measuring and/or quantifying expression of a gene may be found, e.g., in Ausubel et al. (2003 Current Protocols in Molecular Biology, John Wiley & Sons, New York, N.Y.) and in Sambrook et al. (1989 Molecular Cloning: A Laboratory Manual, Cold Spring Harbor Press, Cold Spring Harbor, N.Y.). One skilled in the art will know which parameters may be manipulated to optimize detection of the mRNA or protein of interest.

In particular, the step of measuring the expression of the HypBiomarkers may be done by detecting nucleic acids by a hybridization method (such as a method based on the use of an in situ hybridization microarray), an amplification method (such as quantitative real time PCR), and flow cytometry (such as a method based on the use of nucleic acid-coupled microspheres, e.g. Luminex® microspheres). Detecting nucleic acids can for example be carried out by hybridization using a nucleic acid microarray, by amplification by quantitative real time PCR, by flow cytometry using nucleic acid-coupled microspheres, or by in situ hybridization.

In a preferred embodiment, a nucleic acid microarray is used to measure the expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.).

In another preferred embodiment, quantitative real-time PCR (qRT-PCR) is used to measure the expression of the HypBiomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green. The action may also be performed with a fluorescent reporter probe that is specific for the DNA being amplified. A non-limiting example of a fluorescent reporter probe is a TaqMan® probe (Applied Biosystems, Foster City, Calif.). To minimize errors and reduce any sample-to-sample variation, qRT-PCR is typically performed using a reference standard. The ideal reference standard is expressed at a constant level among different tissues, and is unaffected by the experimental treatment. Suitable reference standards include, but are not limited to, mRNAs for the housekeeping genes coding for ribosomal protein L32 (RPL32) and ribosomal RNA (18S). The level of mRNA in the original sample or the fold change in expression of each biomarker may be determined using calculations well known in the art.

A “biological sample” in accordance with the invention comprises or consists of tumor cells or tumor tissues. The cells may for example be fresh, frozen, fixed or embedded cells. Methods for obtaining such cells and tissues are well-known to the skilled in the art and include, e.g., needle aspiration, incisional biopsy and surgical resection.

In a specific embodiment of the invention, the invention provides the quantification of the listed biomarkers (HypBiomarkers) in an ex vivo experiment where tumor cells or tissues extracted from patients are cultivated in a hypoxic atmosphere, or with a hypoxia mimetic compound such as cobalt and/or dimethyl oxalyl glycine, or with a therapeutic compound in order to predict treatment susceptibility (e.g. radiation or chemotherapy susceptibility) of the patient. Such a method is useful for screening for efficient treatments for a given patient. Indeed, once the tumor cells of the patients have been taken, one can cultivate these tumors cells and determine treatments to which the patient is likely to respond. In addition, a migration assay may then be carried out.

Therefore, the biological sample comprising tumor cells or tumor tissues may be cultivated ex vivo before carrying out step (a) of the methods according to the invention. Said cultivation may be carried out in hypoxic conditions (i.e. in a hypoxic atmosphere or with a hypoxia mimetic compound) and/or in the presence of a therapeutic compound.

3. Kits According to the Invention

Another aspect of the invention pertains to a kit for predicting the outcome, the survival and/or the response of a patient suffering from cancer, preferably breast cancer, said kit comprising or consisting of means for measuring the expression of at least 5, 10, 15, 20, 25, 30, 35, 40 or 45 of the HypBiomarkers according to the invention. The kit may further comprise means for measuring expression of other genes known to be predicting for the outcome, the survival and/or the response of a patient suffering from cancer, preferably breast cancer.

The kit may further comprise, e.g.:
means for converting the expression of each biomarker into an expression value;
means for analyzing the expression values to generate a risk score that predicts the risk of relapse;
reagents for carrying out the measure of expression (e.g. a polymerase and/or an qRT-PCR reaction mix); and/or
instructions for use of the kit for predicting the risk of relapse, outcome, the survival and/or the response of a patient suffering from cancer, preferably breast cancer.

The kit according to the invention may for example comprise or consist of an array (e.g. a microarray) comprising said means for measuring expression.

A further aspect of the invention is thus directed to an array or microarray comprising means for measuring the expression of at least 5, 10, 15, 20, 25, 30, 35, 40 or 45 of the HypBiomarkers according to the invention.

Means for measuring expression of a gene are well-known in the art and include, e.g., polynucleotides suitable for measuring expression of said gene by hybridization and/or by amplification, and antibodies specifically binding to the protein encoded by the gene.

The means for measuring expression of a gene may for example comprise or consist of polynucleotides such as primers and probes. Polynucleotides suitable for measuring expression of HypBiomarkers can easily be obtained by the skilled in the art.

In a preferred embodiment, the polynucleotides suitable for measuring expression of HypBiomarkers consist of primers and probes for use in qRT-PCR.

A “polynucleotide” refers to the phosphate ester polymeric form of ribonucleosides (“RNA molecules”) or deoxyribonucleosides (“DNA molecules”), or any phosphoester analogs thereof, such as phosphorothioates and thioesters, in either single stranded form, or a double-stranded helix.

The term “primer” is meant for short nucleic acid molecules, such as a DNA oligonucleotide, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule. Primer pairs can be used for amplification of a nucleic acid sequence, for example, by PCR, real-time PCR, or other nucleic-acid amplification methods known in the art. Methods for preparing and using primers are described for example, in Sambrook et al. (1989 Molecular Cloning: A Laboratory Manual, Cold Spring Harbor, N.Y.).

The term “probe” refers to an isolated nucleic acid capable of hybridizing to a target nucleic acid. A detectable label or reporter molecule can be attached to a probe. Typical labels include radioactive isotopes, enzyme substrates, co-factors, ligands, chemiluminescent or fluorescent agents, haptens, and enzymes. Methods for labelling and guidance in the choice of labels appropriate for various purposes are discussed, for example, in Sambrook et al. (1989 Molecular Cloning; A Laboratory Manual, Cold Spring Harbor).

Primers and probes are preferably at least 12, 15, 20, 25, 30 or 50 nucleotide long.

Primers and probes can be, e.g., less than 500, 250, 200, 150, 100, or 50 nucleotide long.

Such primers and probes are well-known in the art. For example, the primers and probes suitable for measuring expression of HypBiomarkers may comprise or consist of a fragment of the sequences listed in Table 1, or of the sequence complementary thereto. Alternatively, they may comprise of consist of a fragment of sequence at least 80, 85, 90, 95, 97 or 100% identical to the sequences listed in Table 1, or to the sequence complementary thereto. Said fragment may be a fragment of at least 12, 15, 20, 25, 30, 50, 100, 150, 200, 250, 300, 350, 400, 450 or 500 nucleotides of said sequence.

“Percentage of identity” between two nucleic acid sequences is intended to mean the percentage of nucleic acid which are identical in the two sequences to be compared. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used for determining the percentage of identity. The needle program is for example available on the ebi.ac.uk world wide web site.

4. Uses of the HypBiomarkers According to the Invention

An additional aspect is a plurality of biomarkers for use in predicting survival of an individual with cancer, preferably breast cancer.

In particular, the invention pertains to the use of at least 5, 10, 15, 20, 25, 30, 35, 40 or 45 of the HypBiomarkers according to the invention as biomarkers for predicting outcome of cancer, preferably breast cancer, for predicting survival and/or response of a patient suffering from cancer, preferably breast cancer, and/or for designing a treatment regimen for a patient suffering from cancer, preferably breast cancer.

The invention also pertains to the use of for measuring the expression of at least 5, 10, 15, 20, 25, 30, 35, 40 or 45 HypBiomarkers for predicting outcome of cancer, preferably breast cancer, for predicting survival and/or response of a patient suffering from cancer, preferably breast cancer, and/or for designing a treatment regimen for a patient suffering from cancer, preferably breast cancer.

The present invention will be more readily understood by referring to the following examples. These examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

### BRIEF DESCRIPTION OF THE FIGURES

FIG. 1. A. Percentage of relapse among patients having either a relapse score according to the invention that is inferior or equal to 2, or a risk score superior to 2. Curve 1: 15 patients with a score 2. Curve 2=17 patients with a score of 3+. The 12 relapses were in the curve 2 (p=0.021 Test of Mantel-Haenszel). B. The Cronbach's alpha index is around 0.9.

FIG. 2. Relative Quantification PCR analyses of expression of hypoxic genes from invasive breast tumors. The ratio of the average relative quantification of relapse samples on non relapse samples is represented for different hypoxic genes by a classification in ascending order. Comparison of means using Student's t test (grey bar: P<0.05 and black bars: P<0.1). Underlined genes correspond to genes that are found to be significant using the non parametric Kruskall Wallis test. All HypBiomarkers are overepxressed in relapse samples versus non relapse samples, with a median overexpression of 76%.

### EXAMPLES

### Example 1

### Materials and Methods

1.1. Patients

Forty patients with previously untreated primary invasive ductal breast carcinoma were included in this retrospective study. Initial staging comprised complete and detailed clinical examination including the International Union Against Cancer TNM (tumor size, nodes, metastases) classification. Ultrasound examination and bilateral mammography were also performed. Histopathological evaluation of the tumours was performed on core needle biopsies by Scarff, Bloom and Richardson (SBR) grading as modified by Elston and Ellis. One sample for each patient was used for DNA analysis by flow cytometry with EPICS V (Beckman-Coulter, Roissy, France). The status of oestrogen and progesterone receptors and HER2 were determined by immunohistochemistry on paraffin-embedded sections 3 μm thick. Immunostaining was performed with a Nexes automated immunostainer (Ventana, Illkirch, France). Sections were scored semiquantitatively by light microscopy by two pathologists. For oestrogen and progesterone receptors, a threshold of 10% of stained nuclei was considered positive. For HER2, overexpression corresponded to more than 10% of cells showing complete membrane staining with high intensity. HER2 fluorescence in situ hybridization (FISH) assay was used for detection of amplification of the HER2 gene in paraffin-embedded tissue sections of tumors (HER2 FISH pharmaDx™ Kit Dako Cytomation).

1.2. RNA Extraction and Reverse Transcription

Fine-needle aspirates were performed in patients diagnosed for breast cancer. An aliquot of each aspirate was smeared on a slide to serve as a control, by a pathologist, for the presence of malignant cells and the absence of important stromal and fat contamination. Aspirates were stored in liquid nitrogen until RNA extraction. Total RNA was extracted with Trizol reagent (Gibco/BRL, Cergy-Pontoise, France). RNA was then stored at −80° C. until cDNA synthesis and amplification reaction. The RNA quality was checked by electrophoresis using a Bioanalyzer 2100 with RNA 6000 Nano LabChip® and BioSizing A.02.11 software (Agilent Technologies). Two micrograms of total RNA were reverse transcribed in a total volume of 20 μl using a high capacity cDNA reverse transcription Kit (Applied Biosystems) according to the manufacturer's instructions.

1.3. Assay Design and Real Time PCR

Real-time RT-PCR analysis was performed with custom designed low density arrays in 384-well micro fluidic cards. For each card, there are eight separate loading ports that feed into 48 separate wells for a total of 384 wells per card. Each well contains specific, well-defined primers and probes which were ordered from and designed by Applied Biosystem, and which are capable of detecting a single gene. In this study, the card was configured into eight identical 47-gene sets. Genes were chosen based on their induction by hypoxia and their involvement in breast carcinogenesis literature reviews. The set of 47 genes also contains two housekeeping genes, RPL32 and 18S. In addition, the gene expression stability was determined by the NormFinder program (Andersen et al. Cancer Res 2004; 64:5245-5250) and three optimal normalization genes was identifying among the gene set.

A total of 100 μl reaction mixtures with 50 μl cDNA template (100 ng) and an equal volume of TagMan® universal master mix (Applied Biosystems) was added to each line of TLDA after gentle vortex mixing. The array was centrifuged twice for 1 minute each at 1200 rpm to distribute the samples from the loading port into each well. The card was then sealed and PCR amplification was performed using an Applied Biosystems Prism 7900HT sequence detection system. Thermal cycler conditions were as follows: 2 min at 50° C., 10 min at 94.5° C. and 30 s at 97° C., and 1 min at 59.7° C. for 40 cycles. The threshold cycle Ct was automatically given by SDS2.2 software package. Relative quantities (RQ) were determined using the equation: RQ=2−ΔΔCt.

1.4. Statistics

Student test were used to compare quantitative and categorical parameters. P<0.10 was considered significant. The H test of Kruskal-Wallis was used to compare group of non-relapse versus group of relapse. The internal consistency of different markers group was assessed using Cronbach's alpha coefficient as a measure of scale reliability.

### Example 2

### Results and Calculation of Risk Score of Relapse

Expression of all HypBiomarkers was quantified by RT-PCR using a Taqman low density array (Applied Biosystem). Relative quantities (RQ) were determined for 40 samples of patients suffering from early stage invasive ductal breast carcinoma. Several parameters, including those obtained by performing a Fisher test (F), a Student test (S) or a Kruskall-Wallis test (H), were determined. A comparative analysis of the results between different groups allowed identifying which Hypbiomarkers were significantly expressed between the different groups. A clustering was then carried out using the caGEDA software (expression threshold: 1.5, K-Mean clustering, J5 statistical test). Using a logistic regression model, HyBiomarkers which allowed predicting the status of a patient were identified.

It was found that the EPO, ETS1, ENO1, PGK1, VEGFA, LDHA and TPI markers are significantly overexpressed in the group of patients that relapsed. It was further found that the NHERF1, ERBB2, PTEN, MET, CDH1 and BRCA1 markers are positively correlated with the grade. In addition, it was found that the CATHD, MDR1, NIX, FOXO3, ETS1, TWIST1, VHL, IGF2, PHD2 and EDN1 markers are associated with the progesterone receptor. Regarding the Her2 status, overexpression of the HER2 protein is mainly associated with overexpression of ERBB2 and NHERF1.

In summary, it has been demonstrated that the HypBiomarkers are associated with tumors of high grades. In addition, a coordinated overexpression of HypBiomarkers in HER2+ and RP+ tumors has been shown. Therefore, the HypBiomarkers can be used as markers of poor prognosis and/or poor response to treatment of breast cancers such as invasive ductal breast carcinomas.

To define a risk score of relapse, the optimum expression for each gene, significantly expressed in the relapse group, was determined. More specifically, relative quantities (RQ) were determined using the equation: RQ=2−ΔΔCt.

For each one of the EPO, ETS1, ENO1, PGK1, LDHA and TPI HypBiomarkers, the number of 1 was attributed if its expression was higher than the optimum thresholds presented below in Table 2. The risk score was then calculated by making the sum of the numbers attributed for each one of the EPO, ETS1, ENO1, PGK1, LDHA and TPI HypBiomarkers.

TABLE 2

| Genes | Optima |
|---|---|
| EPO | 7.1 |
| ETS1 | 1.81 |
| ENO1 | 1 |
| PGK1 | 1.37 |
| LDHA | 1.2 |
| TPI | 1.14 |

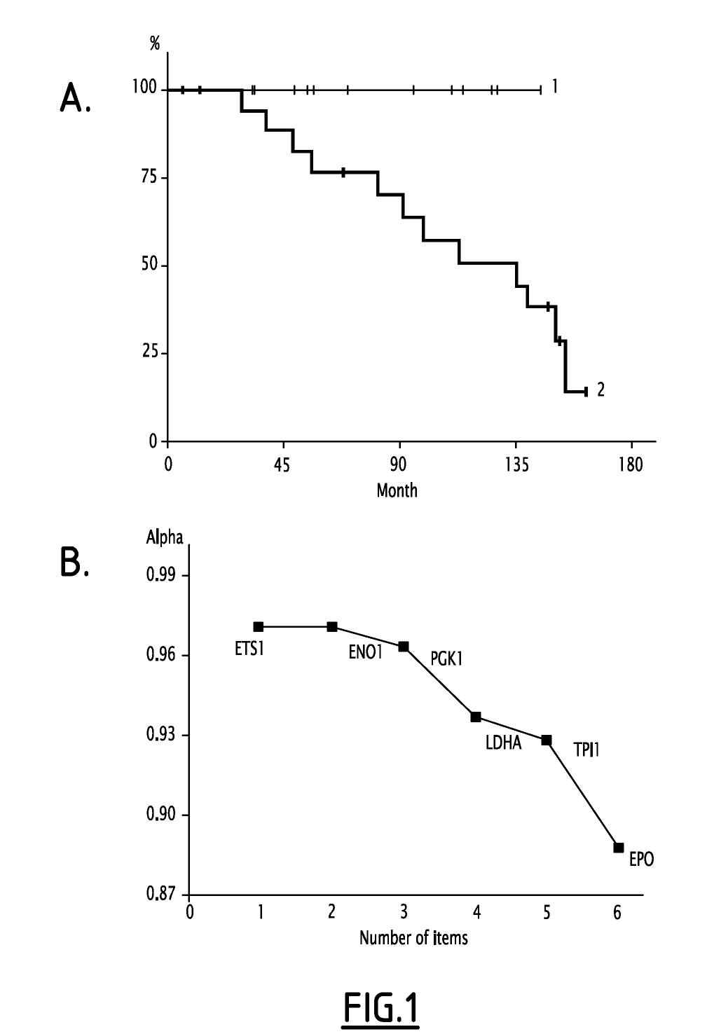

As shown on FIG. 1A, a score threshold to 2 achieves a significant difference between relapse vs. no relapse. The risk of relapse is multiplied by 1.384 if the score is 3, which means a risk of relapse that is increased by 40%. In group with a score 3, the relapse rate after 5 years is 19% and the relapse rate after 10 years is 42%, while it is 0% in the other group because no patient belonging to this group had relapsed. In addition the statistical index of Cronbach's alpha (Cronbach 1951 Psychometrika 16:297-334) indicates that there is a good consistency between all these markers (FIG. 1B).

The analysis of the expression values of EPO, ETS1, ENO1, PGK1, LDHA and TPI allows to generate a risk score of relapse, wherein a risk score having a value 3 is indicative of a short relapse time and a risk score having a value 2 is indicative of a long relapse time. Then, analysis of the expression values of each gene significantly expressed in high grade group and Her2 positive group is associated to outcome and response to treatment. Indeed the overexpression of these genes is shown to be correlated with a poorer prognostic for response, outcome and survival.

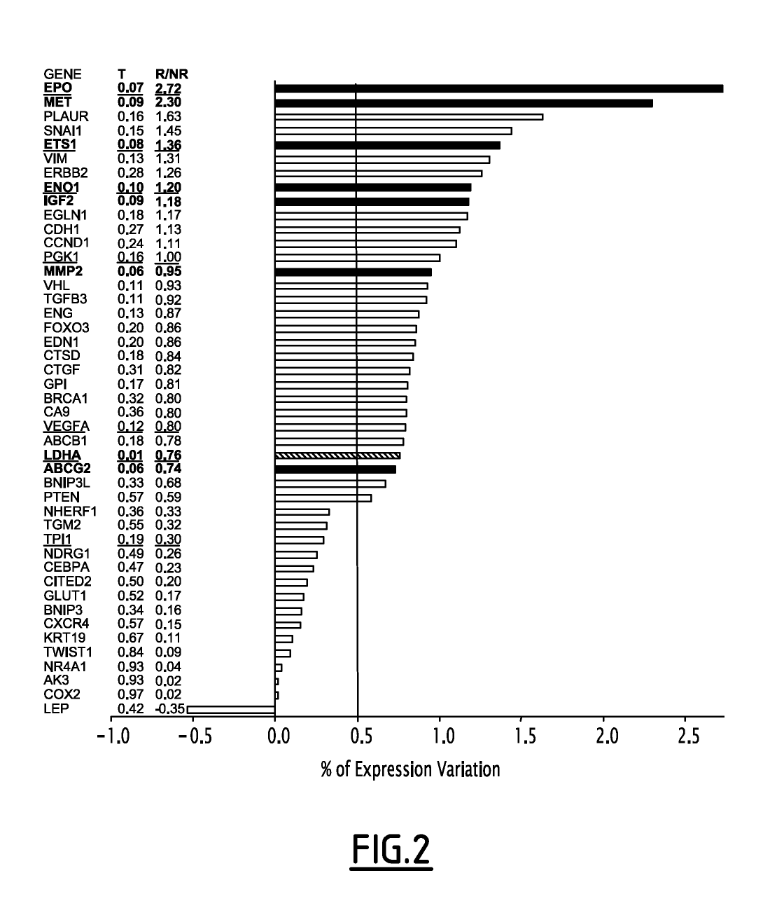

In addition, as shown on FIG. 2, it was found that the HypBiomarkers are overexpressed in patients who have relapsed compared to patients who have not relapsed.

Similar studies were carried out to determine which HypBiomarkers are significantly overexpressed in patients of SBR grade 2-3 versus patients of SBR grade 1, patients of high mSBR grade versus patients of low mSBR grade, RP+ versus RP− patients, Her2 3+2+ versus Her2-Her2 1+ patients, Her2 1+2+3+ versus Her2− patients and Her2 3+ versus Her2− patients (data not shown). It was found that:
NHERF1, ERBB2, PTEN, MET and CDH1 are significantly overexpressed in patients of SBR grade 2-3 versus patients of SBR grade 1;
NHERF1, ERBB2, PTEN, BRCA1 and CDH1 are significantly overexpressed in high mSBR grade versus patients of low mSBR grade;
CATHD, MDR1, NIX, FOXO3A, ETS1, TWIST1, VHL, IGF2, PHD2 and EDN1 are significantly overexpressed in RP+ versus RP− patients;
ERBB2, NHERF1, CDH1, TPI, CITED2, KRT19 and GLUT1 are significantly overexpressed in Her2-positive cancer versus Her2-negative patients;
ERBB2, NHERF1, COX2, TWIST1, PHD2, PGK1, FOXO3A, TGM2, CDH1, CATHD and EDN1 are significantly overexpressed in Her2 3+2+ versus Her2-Her2 1+ patients; and
ERBB2, NHERF1, COX2, PHD2, PGK1, FOXO3A, TPI, MET, PTEN, ENG and GLUT1 are significantly overexpressed in Her2 3+ versus Her2− patients.

1. A method for predicting the risk of relapse of a patient suffering from cancer, said method comprising the steps of:
a) quantifying expression levels of mRNA or protein for at least five genes selected from the group consisting of GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2 in a biological sample of said patient; and
b) comparing the expression levels of mRNA or protein from said at least five genes to corresponding expression levels of said at least five genes in a reference biological sample to generate a risk score of relapse;
wherein a risk score greater than or equal to three is indicative of high risk of relapse and a risk score less than or equal to two is indicative of a low risk of relapse.

2. A method for predicting outcome of cancer, and/or for predicting survival and/or response of a patient suffering from cancer, said method comprising the steps of:
a) quantifying expression levels of mRNA or protein for at least five genes selected from the group consisting of GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2 in a biological sample of a patient;
b) comparing the expression levels of mRNA or protein from said at least five genes to corresponding expression levels of said at least five genes in a reference biological sample; and
c) correlating the expression levels measured at step (a) with outcome, survival, risk of relapse and/or response, thereby deducing the outcome of said cancer, and/or the survival, the risk of relapse or the response of said patient.

3. The method according to claim 1 or 2, wherein said at least five genes comprise EPO, ETS1, ENO1, PGK1, LDHA, TPI and optionally VEGFA, and wherein a significant overexpression of at least three of EPO, ETS1, ENO1, PGK1, LDHA, TPI and optionally VEGFA in said patient, compared to a reference value, indicates that said patient is likely to relapse.

4. The method according to claim 1 or 2, wherein said at least five genes comprise NHERF1, ERBB2, PTEN, CDH1, MET and BRCA1, and wherein a significant overexpression of NHERF1, ERBB2, PTEN, MET and CDH1 or of NHERF1, ERBB2, PTEN, BRCA1 and CDH1 in said patient, compared to a reference value, indicates that said cancer is likely to be an aggressive cancer.

5. The method according to claim 1 or 2, wherein said at least five genes comprise ERBB2, NHERF1, CDH1, TPI, CITED2, KRT19, COX2, TWIST1, PHD2, PGK1, FOXO3, TGM2, CATHD, EDN1, MET, PTEN, ENG and GLUT1, and wherein a significant overexpression of:
i. ERBB2, NHERF1, CDH1, TPI, CITED2, KRT19 and GLUT1;
ii. ERBB2, NHERF1, COX2, TWIST1, PHD2, PGK1, FOXO3, TGM2, CDH1, CATHD and EDN1; or
iii. ERBB2, NHERF1, COX2, PHD2, PGK1, FOXO3, TPI, MET, PTEN, ENG and GLUT1;
in said patient, compared to a reference value, indicates that said cancer is likely to respond to therapies targeting HER receptors.

6. The method according to claim 1 or 2, wherein said step of measuring the expression comprises detecting nucleic acids by a method selected from the group consisting of a hybridization method, an amplification method and flow cytometry.

7. The method according to claim 1 or 2, wherein the biological sample comprises fresh, frozen, fixed or embedded cells tumor cells.

8. The method according to claim 1 or 2, wherein said biological sample comprises tumor cells or tumor tissues of said patient, and wherein:
i. said cells or tissues are cultivated ex vivo before carrying out step (a); and, optionally,
ii. a migration assay is carried out with said cells or tissues.

9. The method according to claim 8, wherein said cultivation is carried out in hypoxic conditions and/or in the presence of a therapeutic compound.

10. The method according to claim 1 or 2, wherein said method comprises predicting response of a patient to therapies targeting HER receptors and/or VEGF receptors.

11. The method according to claim 1 or 2, further comprising the step of designing a treatment regimen for said patient.

12. A kit for predicting outcome of cancer, and/or for predicting survival and/or response of a patient suffering from cancer, said kit comprising an array of polynucleotides for detecting said genes by hybridization or by amplification-for measuring the expression of at least five genes selected from the group consisting of GLUT1, PGK1, LDHA, ENO1, CAIX, NHERF1, TPI, AMF/GPI, VEGFA, TGFB3, ENG, LEP, EDN1, MDR1, AK3, MXR1, TGM2, CDH1, MMP2, CK19, VIM, CXCR4, UPAR, CATHD, CTGF, COX2, MET, IGF2, CCND1, EPO, NDRG1, BNIP3, NIX, ETS1, PHD2, TWIST1, SNAI1, CEBPA, CITED2, FOXO3A, NUR77, BRCA1, PTEN, VHL and ERBB2.

13. (canceled)

14. (canceled)

15. The method according to claim 1 or 2, wherein said cancer is breast cancer.

16. The kit according to claim 12, wherein said cancer is breast cancer.

17. The method according to claim 1 or 2, wherein said cancer is a malignant solid tumor.

18. The method according to claim 1 or 2, wherein said cancer is a hypoxic solid tumor.

19. The kit according to claim 12, wherein said cancer is a malignant solid tumor.

20. The kit according to claim 12, wherein said cancer is a hypoxic solid tumor.
